(19) United States
(12) Patent Application Publication
(10) Pub. No.: US 2022/0330947 A1
(43) Pub. Date: Oct. 20, 2022

(54) IMPLANT FOR THE TREATMENT OF ANEURYSMS

(71) Applicant: femtos GmbH, Bochum (DE)

(72) Inventors: Hans HENKES, Stuttgart (DE); Hermann MONSTADT, Bochum (DE); Ralf HANNES, Dortmund (DE); Volker TRÖSKEN, Witten (DE); Cameron MENZIES, Gladbeck (DE); Ronald KONTEK, Bochum (DE)

(73) Assignee: femtos GmbH, Bochum (DE)

(21) Appl. No.: 17/762,970

(22) PCT Filed: Oct. 14, 2020
(86) PCT No.: PCT/EP2020/078894

§ 371 Date: Mar. 23, 2022

(30) Foreign Application Priority Data
Oct. 17, 2019 (DE) 10 2019 128 102.4

(51) Int. Cl. A61B 17/12

(52) U.S. Cl. CPC A61B 17/12113; A61B 17/12172; A61B 17/12163; A61B 17/12177; A61B 2017/00526

(57) ABSTRACT

An implant (1) for the treatment of aneurysms (11), wherein the implant (1) has an elongated configuration navigable via a microcatheter (12) and a secondary configuration which it assumes upon release from the microcatheter (12). The implant (1) forms an open, unrolled structure (2) extending from proximal to distal, said implant being built up of a plurality of struts (3) forming adjacent cells (4), and with at least a part of said cells (4) being provided with membranes (6) filling the cells (4), and with the secondary structure comprising an at least partial rolling up of the open, unrolled structure (2) axially and radially relative to the longitudinal axis of the implant (1) and resulting in the formation of a spherical structure. The implant (1) proposed by the invention is suitable to adapt to the inner wall of an aneurysm (11) and to fill an aneurysm (11) almost completely.

The invention relates to an implant for the treatment of arteriovenous malformations, in particular aneurysms, with the implant being navigable in an elongated state via a microcatheter to a destination in the blood vessel system of a patient, and a secondary structure being imposed on the implant which it assumes upon release from the microcatheter, and the associated elimination of the external constraint, with the implant being detachably connected to a delivery wire.

Aneurysms are usually saclike or fusiform dilatations of the vessel wall and occur primarily in structurally weakened areas of the vessel wall due to the constant pressure of blood. Accordingly, the inner vessel walls of an aneurysm are thus sensitive and susceptible to injury. As a rule, rupture of an aneurysm leads to significant health impairment, and in the case of cerebral aneurysms, to neurological deficits and even fatalities of patients.

Aside from surgical interventions, in which, for example, the aneurysm is clamped by means of a clip, endovascular methods for the treatment of aneurysms are known in particular, with two approaches being primarily pursued. One option includes filling the aneurysm with occlusion means, especially using so-called coils (platinum spirals) for this. The coils facilitate the formation of a thrombus and thus ensure occlusion of the aneurysm. On the other hand, it is known to close off the access to the aneurysm, for example the neck of an aciniform aneurysm, from the blood vessel side making use of stent-like implants and in this manner disconnect it from the blood flow. Both methods serve to reduce the blood flow into the aneurysm and in this way alleviate, ideally even eliminating the pressure acting on the aneurysm and thus reducing the risk of an aneurysm rupture.

When filling an aneurysm with coils it may happen that the filling of the aneurysm is inadequate, allowing blood flow into the aneurysm and in this way cause the pressure acting on its inner wall to continue. The risk of steady dilation of the aneurysm and eventually its rupture persists, albeit in an attenuated form. Moreover, this treatment method is only suitable for aneurysms having a relatively narrow neck—so-called aciniform aneurysms—as otherwise there is the risk that coils protrude from a wide aneurysm neck into the blood vessel where they produce clots, which may lead to occlusions in the vessel. In the worst case, a coil is completely washed out of the aneurysm and causes vessels to be occluded elsewhere.

To keep the coils in place in the aneurysm sac, the aneurysm neck is often additionally covered with a special stent.

Another intravascular treatment approach focusses on so-called flow diverters. These implants are similar in appearance to stents that are used for the treatment of stenoses. However, since the purpose of the flow diverters is not to keep a vessel open, but to obstruct access to the aneurysm on the blood vessel side, their mesh width is very narrow; alternatively, implants of this kind are coated with a membrane. A disadvantage of these implants is the risk that outgoing side branches in the immediate vicinity of the aneurysm to be treated are sometimes also covered and thus closed off in the medium or long term.

WO 2012/034135 A1 discloses an implant which comprises a first and a second section, which are arranged one behind the other within a catheter, but after release within the aneurysm takes on a three-dimensional, near-spherical shape and thus fills the aneurysm. The basic material for the three-dimensional implant is a mesh-like fabric, the embodiment examples and figures are all based on a tubular braiding consisting of shape memory material. The disadvantage of this prior art has been found to be that said implant has an unfavorable stiffness. Due to the fact that aneurysms are rarely absolutely round, a three-dimensional implant should be able to adapt to the morphology of the aneurysm in the best possible way. Furthermore, the implant is too bulky for low-caliber catheters.

Another implant for insertion into aneurysms is disclosed in WO 2017/089451 A1. The implant described therein comprises several subunits, each of which has a framework of struts with a covering arranged between them. However, the implant must first form into a plurality of coils within an aneurysm until sufficient coverage of the aneurysm surface is achieved.

Proceeding from the state of the art described hereinbefore, the objective is to provide a further improved implant for insertion into aneurysms that in particular ensures good coverage of the aneurysm wall and filling of the interior of the aneurysm.

As proposed by the present invention, this objective is achieved by providing an implant for the treatment of arteriovenous malformations, in particular aneurysms, wherein the implant in an elongated state being navigable via a microcatheter to a destination in the blood vessel system of a patient and a secondary structure being imposed on the implant which it assumes upon release from the microcatheter and the associated removal of the external constraint, wherein the implant is detachably connected to a delivery wire and wherein the implant forms an area structure extending from proximal to distal, said structure being built up of a plurality of struts forming adjacent area or sheet-like segments, and with at least a part of said cells being provided with membranes filling the cells, and with the secondary structure comprising an at least partial rolling up of the area structure axially and radially to the longitudinal direction of the implant and resulting in the formation of a spherical structure.

The implant according to the invention is composed of several struts forming into contiguous cells. These combine to create an area structure that can be spread flat and, in this conformation, results in an elongated element resembling a stent which is longitudinally slit and spread out flat. Similar to this, a secondary structure imposed on the implant ensures that the area structure tends to roll up at least partially radially to the longitudinal direction of the implant, that is, around the longitudinal axis. As a rule, rolling up takes place along the entire length of the area structure. However, the roll-up formation does not have to extend so far as to result in a tube due to the side edges of the area structure overlapping; it is usually sufficient when the above-mentioned secondary structure only causes a partial roll-up of the area structure. In other words, the area structure in this case has side edges that are curved up radially to the longitudinal direction and thus resembles cut-off tree bark. When the implant is inside a microcatheter by means of which it is to be advanced to the target site, the area structure normally exists in rolled-up form, although, depending on the inner diameter of the microcatheter, the area structure may also be rolled up more firmly than predetermined by the secondary structure. In this case, the release from the microcatheter is associated with a widening along and perpendicular to the longitudinal direction of the implant, that is, to some extent, unrolling occurs.

However, rolling up around the longitudinal axis as mentioned before, i.e. radially to the longitudinal direction, is only one aspect of the secondary structure imposed on the implant. In fact, a secondary structure is imposed on the area structure which does not only tend to roll up the structure at least partially radially around the longitudinal axis, but also acts axially, resulting in an overall near-spherical structure. Rolling up in the axial direction is understood to denote a roll-up about an axis that is orthogonal to the longitudinal axis. Accordingly, a roll-up of the area structure takes place in the longitudinal direction of the implant as well as a bending/curvature of the area structure essentially on the longitudinal axis of the implant. As the implant is released from the microcatheter, more of the implant gradually emerges from the microcatheter, with roll-up automatically occurring in such a way as to produce an overall spherical structure, in particular forming an approximately ball-shape configuration. This formation of the spherical structure actually takes place within the aneurysm, with the implant thus being nestled against the inner wall of the aneurysm and filling it. Due to its flexibility, the implant is capable of adapting to different aneurysm shapes and fill them to a large extent, even if they are of a rather irregular shape. Furthermore, the implant is nevertheless pliable enough to rule out injury to the aneurysm wall which is an important requirement because such an injury leads to uncontrolled bleeding and may entail disastrous consequences, for example, in the intracranial region. The spherical structure is normally designed such that it would assume a diameter larger than the interior of the aneurysm during free expansion. Therefore, the implant secures itself in the aneurysm in a force-closed manner.

It is also important, however, that at least part of the cells is provided with membranes, that is, the space between the individual struts forming a particular cell is wholly or partially provided with a membrane spanning the cell. In this way, a largely homogeneous flat surface is created that closely adepts to the inner wall of the aneurysm. Ultimately, a flow modulation at the aneurysm neck is achieved that virtually disconnects the aneurysm completely from the regular flow of blood.

Within the meaning of the invention, a spherical structure is understood to be a structure that has an approximately spherical or an ovoid shape which may as well be somewhat irregular, i.e., do not necessarily be of perfect spherical shape, for example. The spherical structure is three-dimensionally round and suitable for filling an aneurysm, although the structure that in fact forms naturally depends also on the exact shape of the aneurysm.

A significant advantage over the state of the art with respect to the use of coils to fill aneurysms is that coils are prevented from escaping from the aneurysm and thus cause an occlusion of a blood vessel elsewhere. Moreover, the secondary structure ensures an almost complete filling of the aneurysm and thus causes a safe and rapid occlusion.

The aneurysm may be occluded by one or more inventive implants. Likewise, it is also conceivable to combine the use of the implant with other aneurysm treatment methods, for example, by additionally inserting other implants such as coils, by an additional deposition of liquid embolic agents, or by placing a flow diverter or stent in front of the aneurysm.

Wherever the present invention refers to membranes in the plural, it is hereby clarified that there need not be any separation between the individual membranes, which means, the individual membranes may in fact merge into one another and thus form an overall membrane. However, a single membrane is understood to be the area of the overall membrane that fills or spans a specific cell.

Within the meaning of the present invention, a membrane is a thin structure having a planar surface, regardless of whether said structure is permeable, impermeable or partially permeable to liquids. However, to accomplish the aneurysm treatment objective, membranes are preferred that are completely or at least substantially impermeable to fluids such as blood. Moreover, a membrane may also be designed to comprise pores through which further occlusion agents can be introduced. Another option is to have the membrane designed in such a way that it can be pierced with a microcatheter for the introduction of further occlusion agents or even with the occlusion agents themselves.

The membranes can be made of polymer fibers or films, with the membranes being preferably produced by an electrospinning process. In this process, the struts are normally embedded in the membrane. This can be brought about by first creating the area structure, around or over which fibers are then spun or braided in such a way as to produce an area structure whose individual cells are provided or covered with a membrane.

In electrospinning, fibrils or fibers are separated from a polymer solution and deposited on a substrate by applying an electric current. Said deposition causes the fibrils to agglutinate into a non-woven fabric. Usually, the fibrils have a diameter ranging between 100 and 3000 nm. Membranes created by electrospinning have a very uniform texture. The membrane is tenacious, withstands mechanical stresses, and can be pierced mechanically without an opening so created that may give rise to cracks propagating from it. Thickness of the fibrils as well as the degree of porosity can be controlled by selecting appropriate process parameters. In the context of producing the membrane and with respect to materials suitable for this purpose, special attention is drawn to publications WO 2008/049386 A1, DE 28 06 030 A1 and literature referred to therein.

In lieu of electrospinning, the membranes may also be produced by an immersion or spraying process such as spray coating. As regards the material of the membranes, it is important that they are not damaged by the mechanical stresses arising when the implant is drawn into a microcatheter, deployed, unfolded, etc. The membranes should therefore have sufficient elasticity.

The membranes can consist of a polymer material such as polytetrafluoroethylene, polyester, polyamides, polyurethanes or polyolefins. Especially preferred are polycarbonate urethanes (PCU). In particular, an integral connection of the membranes with the area structure is desirable. Such an integral connection can be achieved by covalent bonds provided between the membranes and the area structure. The formation of covalent bonds is promoted by silanization of the area structure, that is, by a chemical bonding of silicon, in particular silane, compounds to at least portions of the surface of the area structure. On surfaces, silicon and silane compounds attach, for example, to hydroxy and carboxy groups. Basically, aside from silanization, other methods of mediating adhesion between the area structure and membranes are also conceivable.

Silane compounds in this context are to be seen as all those compounds which follow the general formula RₘSiXₙ (m, n=0-4, where R stands for organic radicals, in particular alkyl, alkenyl or aryl groups, and X stands for hydrolyzable groups, in particular OR, OH or halogen, with R=alkyl, alkenyl or aryl). In particular, the silane may have the general formula RSiX₃. Moreover, relevant compounds having several silicon atoms also count among the silane compounds. In particular, silane derivatives in the form of organosilicon compounds are regarded as silane compounds in this context.

Additional substances promoting thrombogenesis or endothelial formation may be embedded in or deposited on the membranes. Substances that promote thrombogenesis are therefore advantageous because they support the formation of a thrombus or clot within the aneurysm, which ensures permanent occlusion of the aneurysm. An example in this context are nylon filaments. Because aneurysms are due to degenerative diseases of the vascular wall, particularly atherosclerosis, promoting endothelial formation and correcting endothelial dysfunction may also have beneficial effects. This applies especially to the area where the aneurysm is in contact with the flow of blood in the actual blood vessel (parent vessel). Preferably, substances promoting thrombogenesis are applied to the inner side of the membrane, whereas substances promoting endothelial formation are applied to the outer side of the membrane, with outer side being understood here to denote the side of a membrane facing the vessel wall in the implanted state and the inner side being understood to mean the side of a membrane facing the interior of the aneurysm. Examples of substances that promote thrombogenesis include collagens, whereas, for example, hyaluronic acid, statins (3-hydroxy-3-methylglutaryl coenzyme A reductase inhibitors), and other polymers may promote endothelial cell colonization. Polysaccharides, especially glycosaminoglycans, which are able to mimic the glycocalyx, are particularly suitable polymers. Another material that can be employed is POSS-PCU (polyhedral oligomeric silsesquioxane poly(carbonate-urea) urethane), which is a nanocomposite that has been described, inter alia, as an interstitium for artificial organs and as a coating for medical devices (Tan et al., Crit Rev. Biomed Eng. 2013; 41(6): 495-513). It is also possible to use POSS-PCL (polyhedral oligomeric silsesquioxane poly(caprolactone-urea) urethane). Applicable to both POSS-PCU and POSS-PCL is that in particular functionalized derivatives of these nanocomposites can also be employed. This applies in particular to those derivatives that can be obtained by linking with polyacrylic acid (poly-AA). As POSS-PCU and/or POSS-PCL nanocomposite polymers are only poorly suited for direct immobilization on the surface of an implant, it has been found advantageous to combine polymers such as polyacrylic acid (poly-AA) with the nanocomposite. This can be achieved, for example, by plasma polymerization of acrylic acid. A poly-AA-g-POSS-PCU surface obtained in this way promotes collagen bonding (especially collagen type 1) and thus endothelial formation (cf. Solouk et al., Mater Sci Eng C Mater Biol Appl. 2015; 46: 400-408). Some additives, for example collagen or hyaluronic acid, are also advantageous because they can improve the friction of the tubular sheath against, for example, the inside of a catheter during advancement and the biocompatibility of the implant.

The terms “proximal” and “distal” are to be understood in such a way that, when the implant is inserted, parts facing towards the attending physician are referred to as proximal and parts facing away from the attending physician are referred to as distal. Typically, the implant is thus moved forward in distal direction by means of a microcatheter. If reference is made to the longitudinal direction of the implant this means the direction from proximal to distal. For example, the microcatheter may be a microcatheter having an inside diameter of 0.021″ or 0.027″. The term “axial” refers to the longitudinal axis of the implant, which extends from proximal to distal when the implant is in an elongated state, with “radial” referring to a direction orthogonal to this.

Expediently, at least some cells are provided with center struts, each of which extending from one edge region of a cell to another, usually opposite, edge region of the same cell. These center struts aid in shaping the implant and, in particular, in bringing the membranes in contact with the inner wall of the aneurysm. Furthermore, the thrusting stability of the implant is also improved. The center struts do not need to extend in a straight line from one edge region of a cell to another edge region; it is rather preferred if the center struts are provided with one or more curvatures so that a certain flexibility is maintained with respect to the length of the center struts. This is significant in the context of rolling up the area structure.

The struts and center struts can form individual nodes or points of intersection, with a cell terminating at each of said intersections. The number of struts/center struts, each approaching the node from one direction and meeting at the nodes, typically ranges between 2 and 4. A further cell can start at a node point in each case, but there may also be a short intermediate area that extends between the node point of a cell and the adjacent node point of the distally next cell.

The individual cells are preferably offset from one another in the longitudinal direction. Viewed from proximal to distal, adjacent to a given cell, the next cell may similarly be offset proximally and distally, whereas the next but one cell again lies on the same line parallel to the longitudinal axis of the implant. However, the cell arranged offset from the first cell may also be situated on a line oriented parallel to the longitudinal axis of the implant, together with other cells. When the area structure is spread out flat, a maximum number of two cells are preferably arranged next to each other, where ‘next to each other’ refers to the direction orthogonal to the longitudinal axis.

The size of the individual cells may vary across the implant. The size of the cells may increase in particular from distal to proximal. It is ensured in this way that the distally situated cells, which are the first to exit the microcatheter when it is deployed into the aneurysm, can roll up as tightly as possible. On the other hand, cells situated more proximal wrap around the initially rolled-up distal cells and for that reason exhibit less curvature in longitudinal direction. Moreover, it is also possible for the size of the individual cells to vary orthogonally to the longitudinal direction, for example, cells arranged proximally and distally offset between two cells lying one behind the other in the longitudinal direction have a different size than the two cells arranged one behind the other. Where reference is made to the size of the cells, this refers to the area of the cells with the area structure of which being spread out flat.

To take due consideration of the fact that the distally located cells that are first exiting the microcatheter must curve more than the subsequently exiting cells, it is considered expedient to design the secondary structure in such a way that during the formation of the spherical structure the curvature of the area structure increases from proximal to distal. This increased curvature is already imposed on the implant in the course of the manufacturing process. Furthermore, the spherical structure also conforms to the shape of the aneurysm.

The secondary structure may be designed so as to allow the individual cells in the spherical structure to overlap to a certain degree. This ensures that the entire surface of the inner wall of the aneurysm is covered by the cells and the membranes filling the cells. Where considered appropriate it may also be sufficient to allow for smaller gaps remaining between the individual cells in the spherical structure, as long as at least a comprehensive coverage of the surface of the inner wall of the aneurysm is ensured.

Providing the cells with a membrane is considered to be of particular significance with respect to the cells that will ultimately come to rest on the outer surface of the spherical structure. In contrast, membranes may also be completely or partially dispensed with in the case of cells that are located within the interior of the spherical structure after formation of the latter is completed. It is of course also possible to provide for the entire area structure to be covered with membranes spanning all the cells, with the latter offering advantages particularly for the production process.

Upon full release of the implant, the spherical structure typically has a diameter in the range of between 4 and 25 mm. Such a diameter is sufficient to fill typical aneurysms, especially those located in the intracranial region. The diameter actually created within the aneurysm may vary, allowing the implant to be employed for the treatment of differently sized aneurysms and those with different sizes of the aneurysm neck.

Preferably, the struts of the implant are at least partially made of shape memory materials. This enables the desired secondary structure to be imposed on the implant, which it automatically assumes when leaving the microcatheter. Shape memory metals in particular are well known in the field of medical engineering, with nickel-titanium alloys, such as those used under the name of Nitinol, deserving special mention in this respect.

In particular, the struts of the implant can be produced by laser cutting techniques. Conceivable as well is however to fabricate the area structure composed of the struts in the form of a braided structure, in which individual struts are interwoven or woven together to form the desired area structure as a whole. Other manufacturing processes may be adopted as well, such as galvanic or lithographic production, 3D printing or rapid prototyping. The struts provided may have a round, oval, square or rectangular cross section, with the edges may have a rounded off configuration in the event of a square or rectangular cross section. The individual struts can also be made up of several individual filaments that are twisted together or extending in parallel.

Expediently, the implant is provided with one or several radiopaque markers allowing the attending physician to visualize the treatment. These, for example, may consist of a helix, spiral or rivet made of a radiopaque material, which are attached to struts of the implant. The radiopaque markers may, for example, consist of platinum, palladium, platinum-iridium, tantalum, gold, tungsten or other metals opaque to radiation. It is also possible to provide the implant, in particular the struts or wires of the supporting structure with a coating consisting of a radiopaque material, such as a gold coating. This coating can, for example, have a thickness of 1 to 6 μm. Coating with a radiopaque material need not be applied to the entire supporting structure. Nevertheless, even when applying a radiopaque coating it is considered useful to arrange one or several radiopaque markers on the implant, in particular at the distal end of the implant.

Another approach to render the implant radiopaque would be to embed radiopaque substances, for example heavy metal salts such as barium sulfate, in the membrane. Such substances are known, for example, as contrast agents in X-ray technology applications.

Another option includes using struts made of a metal having shape memory properties, especially an appropriate nickel-titanium alloy, which at least in part comprise a platinum core. Such struts are known as DFT (drawn filled tubing) wires. In this way, the advantageous properties of nickel-titanium on the one hand, namely imparting shape memory properties, are combined, on the other hand, with the beneficial properties offered by platinum, namely ensuring X-ray visibility.

Via a severance point the implant is detachably connected to a delivery wire. This delivery wire may be a conventional guidewire used to advance the implant through the blood vessel system to the desired site. Detachment of the implant from the delivery wire takes place electrolytically, thermally, mechanically or chemically. The preferred electrolytic detachment method provides for the severance point to be electrolytically corroded by applying a voltage causing the implant to disconnect from the delivery wire. To avoid anodic oxidation of the implant, it should be electrically isolated from the severance point and the delivery wire. The electrolytic detachment of implants is well known practice in the state of the art, especially for coils used for the purpose of closing off aneurysms. Relevant severance/detachment points are described, for example, in WO 2011/147567 A1. The principle is based on the fact that when a voltage is applied, a suitably designed severance point made of a suitable material, in particular metal, is dissolved as a rule by anodic oxidation at least to such an extent that the areas of the implant located distally to the corresponding severance point are released. The severance point can be made, for example, of stainless steel, magnesium, magnesium alloys or a cobalt-chromium alloy. The dissolution of the severance point is brought about by applying an electrical voltage. This can be either alternating current or direct current, with a low current intensity (<3 mA) being sufficient. The severance point in this case usually functions as anode the metal of it being oxidized and dissolved. It is important for the severance point to be electrically connected to a voltage source, in particular via the delivery wire. For this purpose, the delivery wire itself must also be of electrically conductive design. Due to the fact that the corrosion-inducing current is influenced by the surface of the cathode, said cathode surface should be significantly greater than the surface of the anode. To a certain extent the speed at which the severance point is dissolved can be controlled by appropriately sizing the cathode surface in relation to the anode surface. Accordingly, the invention also relates to a device comprising a power source and, where applicable or appropriate, an electrode to be placed onto the body surface.

In the event of a mechanical detachment/severance, there is typically a form-closed fit that is broken when the implant is released, causing the implant to be separated from the delivery wire. Another option is to design and provide the severance points in the form of thermal detachment points. When a thermal severance point is provided, the connection between longitudinally adjacent sections of the implant can be broken by heating the severance point, for example a polymer thread, causing it to soften or melt so that detachment is effected. Another option is to make use of chemical severance in such a way that the detachment is brought about by a chemical reaction occurring at the point of detachment.

The different types of detachment, for example electrolytic and mechanical detachment, can also be combined. For this purpose, a mechanical connection, in particular brought about by a form-fit, is established between the elements, and this connection is maintained until an element that keeps up the mechanical connection is electrolytically corroded.

In addition to the implant itself, the invention also relates to the use of the implant for the treatment of arteriovenous malformations, in particular aneurysms, as well as to a method of manufacturing the implant. To this effect, an area structure is first created using a plurality of struts, said structure being configured as described above. Following this, the area structure is made into a spherical structure, subjected to heat treatment and finally provided with membranes. The transformation into a spherical structure can be done, for example, by placing the area structure over a sphere and passing it around the sphere. The finished implant can finally be inserted into a microcatheter; when exiting the catheter, the implant tends to automatically reassume the previously imposed secondary structure.

All statements made with reference to the implant itself also apply in an analogous manner to the use of the implant, a method of using the implant and to the method of manufacturing the implant, with the same applying vice versa.

Further elucidation of the invention is provided by way of examples through the enclosed figures. It should be noted that the figures show preferred embodiment variants of the invention, with the invention itself not being limited thereto. To the extent it is technically expedient, the invention comprises, in particular, any optional combinations of the technical features that are stated in the claims or in the description as being relevant to the invention.

Clarification of the invention is provided by the following figures where

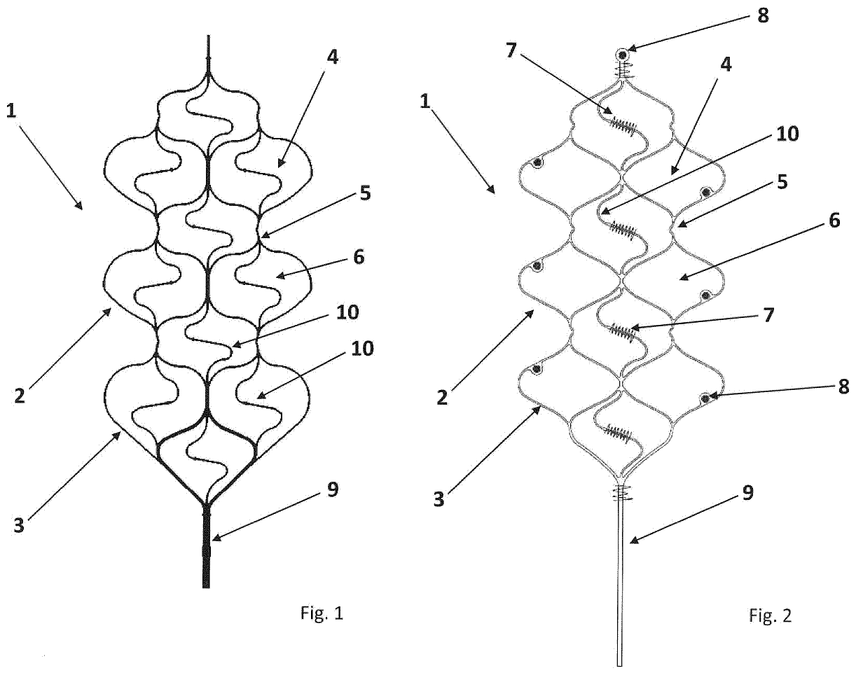

FIG. 1 shows an implant according to the invention in a flat spread out form;

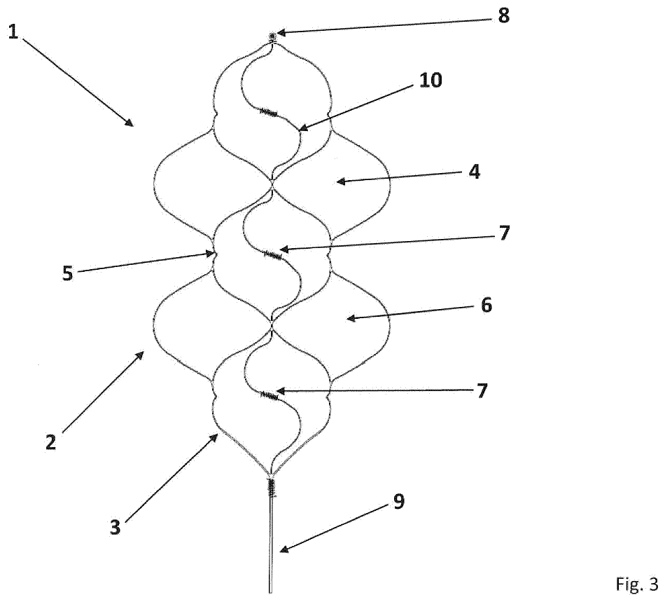

FIG. 2 shows another implant according to the invention in flat spread out form provided with radiopaque markers;

FIG. 3 shows another implant according to the invention in flat spread out form provided with radiopaque markers; and

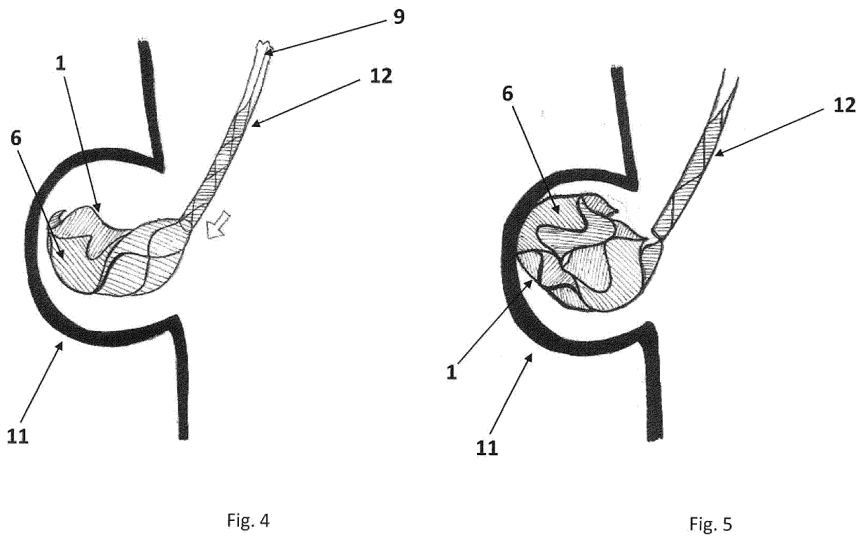

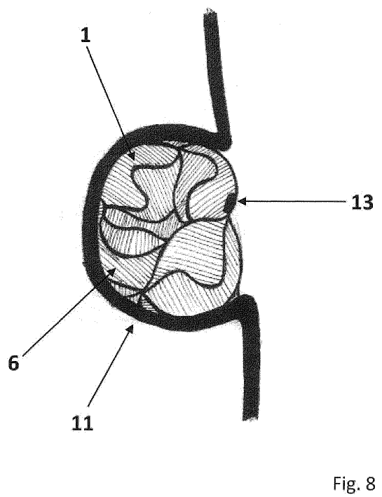

FIGS. 4-8 illustrate the insertion of the inventive implant into an aneurysm.

FIG. 1 shows in a flat spread-out form an implant 1 according to the invention, as well as the area structure 2 which consists of individual struts 3. Struts 3 form various cells 4 arranged offset from one another, with the struts 3 each converging at nodes 5 arranged between the cells 4. The cells 4 are provided with membranes 6. In addition, the individual cells 4 are improved in shape by center struts 10, which at the same time increase the thrusting stability when advancing the implant 1 from proximal to distal. At the proximal end of the implant 1 shown here at the bottom, the implant 1 is attached to a delivery wire 9.

FIG. 2 illustrates an alternative embodiment of the implant 1 proposed by the invention, which is largely similar to the first embodiment. It can be seen that the struts 3 also form cells 4, which are provided with membranes 6, with the totality of the cells 4 forming the area structure 2. Other than shown in the first embodiment, only some cells 4 are provided with center struts 10 in this case. Furthermore, the implant 1 is also attached to a delivery wire 9 at the proximal bottom end of the implant 1.

FIG. 2 additionally indicates radiopaque markers, on the one hand in the form of radiopaque marker spirals 7 and on the other hand in the form of radiopaque rivets 8, with these markers being arranged at various locations on the implant 1 to enable the attending physician to visualize them.

FIG. 3 shows a further embodiment, with the implant 1 in this case also being connected to the delivery wire 9 at the proximal bottom of the implant. Some of the cells 4 are provided with center struts 10, with radiopaque marker spirals 7 being arranged around the center struts 10. Additionally located at the distal top end of implant 1 is a radiopaque rivet 8.

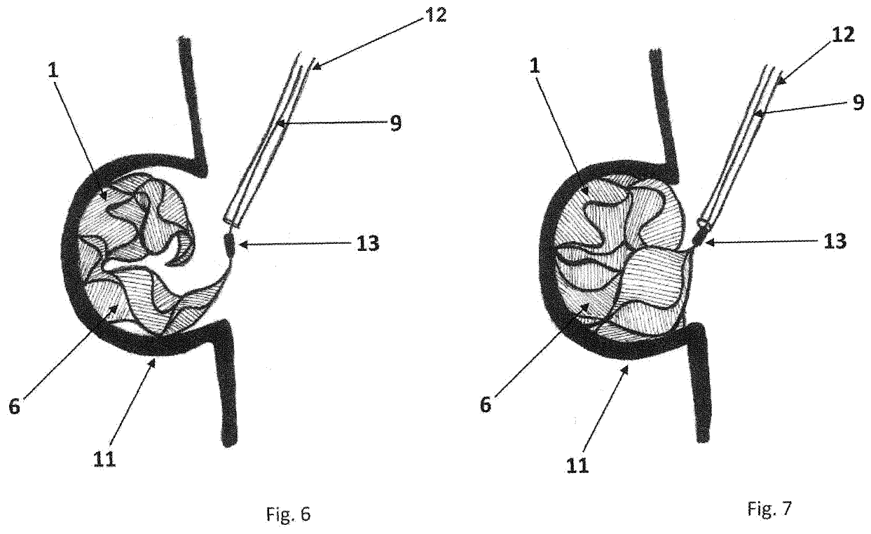

In FIGS. 4 to 8 it is depicted how implant 1 is inserted into an aneurysm 11. By means of the delivery wire 9 the implant 1 is pushed out of and through the microcatheter 12 placed in front of the aneurysm 11 which enables the implant to unfold within aneurysm 11. The implant 1 is provided with membranes 6. During deployment from the microcatheter 12, the implant 1 rolls up radially on the one hand, but also axially, so that it constitutes a spherical structure which occupies the aneurysm 11 practically completely. As the filling process steadily progresses, more of the implant 1 is pushed out of the microcatheter 12, as can be seen in FIGS. 5 to 7. When the implant 1 has been completely pushed out of the microcatheter 12 and the aneurysm 11 is filled completely, a preferably electrolytic detachment can take place at the severance point 13, which forms the connection between the implant 1 and the delivery wire 9. As can be seen in FIG. 8 the implant 1 has been fully inserted into the aneurysm 11 and detached from the delivery wire.

1. An implant for the treatment of aneurysms (11), wherein
the implant (1) has a proximal end and a distal end, said implant comprising a plurality of struts (3) forming adjacent cells (4), with at least a part of said cells (4) being provided with membranes (6) filling the cells (4),
said implant having a first, small diameter, long length configuration in which it is rolled up about its longitudinal axis suitable for placement in a lumen of the microcatheter, and having a second, large diameter, short length configuration in which it is more loosely rolled about its longitudinal axis compared to the first, small diameter, long length configuration and in which it is rolled up relative to a radial axis to comprise a balled-up configuration adapted for disposition with a saccular aneurysm.

2. An implant according to claim 1, wherein:
the membranes (6) comprise polymer fibers or polymer films.

3. An implant according to claim 1, wherein:
the membranes (6) are produced by an electrospinning process.

4. An implant according to any one of claim 1, wherein:
the membranes (6) are composed of an electrospun polycarbonate urethane.

5. An implant according to claim 1, wherein:
the membranes (6) comprise substances promoting endothelial formation and/or thrombogenesis.

6. An implant according to claim 1, wherein:
at least some of the cells (4) are provided with center struts (10) each extending from one edge region of a cell (4) to another edge region.

7. An implant according to claim 1, wherein:
the individual cells (4) are arranged offset from one another in the longitudinal direction.

8. An implant according to claim 1, wherein:
the size of the cells (4) varies.

9. An implant according to claim 8, wherein:
the size of the cells (4) increases from the distal end to proximal end of the implant.

10. An implant according to claim 1, wherein:
the curvature of the implant (2), when in the second, large diameter, short length configuration increases from the proximal end to distal end thereof.

11. An implant according to claim 1, wherein:
when in the second, large diameter, short length configuration the individual cells (4) in the implant overlap.

12. An implant according to claim 1, wherein:
at least a part of the cells (4), which are located in the interior of the implant after its formation, are not or only partially provided with a membrane (6).

13. An implant according to claim 1, wherein:
the implant has a diameter ranging between 4 and 25 mm when the implant (1) has been fully released.

14. An implant according to claim 1, further comprising radiopaque markers (7, 8).

15. A method for manufacturing an implant according claim 1, wherein the implant (2) is created from a plurality of struts (3) and the implant (2) is transformed into a balled-up configuration adapted for disposition within a saccular aneurysm, subjected to heat treatment and provided with membranes (6).

16. An implant according to claim 1, wherein:
the implant is configured for detachable connection to a delivery wire (9).
